(19) United States
(12) Patent Application Publication
(10) Pub. No.: US 2024/0423578 A1
(43) Pub. Date: Dec. 26, 2024

(54) GEOMETRIC CALIBRATION IN AN X-RAY IMAGING SYSTEM

(71) Applicant: 3DIO, Inc., Orem, UT (US)

(72) Inventors: Douglas P. Hansen, Spanish Fork, UT (US); Jacob Cassinat, Provo, UT (US); Kolby Black, Salem, UT (US); Mathew Beardall, Provo, UT (US); Jean-Dominique Gascuel, Meylan (FR)

(21) Appl. No.: 18/337,719

(22) Filed: Jun. 20, 2023

(51) Int. Cl. A61B 6/00; A61B 6/14

(52) U.S. Cl. CPC A61B 6/583; A61B 6/51

(57) ABSTRACT

Systems and methods for the calibration of X-ray devices and systems are described in this application. In particular, this application describes a calibration apparatus for an X-ray imaging system that comprises a housing containing an X-ray sensor and a calibration array having multiple calibration targets located within or on a substrate. Each calibration target comprises an element with an X-ray absorption higher than that of the material of the substrate. The multiple targets are in a fixed spatial relationship relative to each other. The calibration apparatus can be incorporated into an aligner that is used with the X-ray imaging system. That calibration apparatus increases the accuracy of the construction of a three dimensional (3D) image from a series of two dimensional (2D) images taken by the X-ray imaging system. Other embodiments are described.

### FIELD

This application relates generally to X-ray equipment. More specifically, this application relates to X-ray devices and systems that are used for calibration during the reconstruction of a three dimensional (3D) image from a series of two dimensional (2D) images.

### BACKGROUND

X-ray imaging systems typically contain an X-ray source and an X-ray detector. X-rays are emitted from the source and impinge on the X-ray detector to provide an X-ray projection image, or a shadow image, of the object or objects that are placed between the X-ray source and the detector. The X-ray detector is often an image intensifier or even a flat panel digital detector. X-ray imaging systems have been developed that produce either 2D or 3D images. The imaging systems that produce 3D images typically employ computed tomography techniques to reconstruct a 3D image from multiple 2D images.

When patients are subject to X-rays, particularly in dental applications, involuntary patient movement and other sources of shot-to-shot variation can lead to errors in constructing a 3D image from the series of 2D images. Traditionally, this patient movement can be dealt with by immobilizing the patient through bite-blocks, head rests, or other means of providing a physical restraint, or through a calibration process that determines the geometry of the X-ray source and the detector at the moment the data is acquired. Since the calibration must account for shot-to-shot variations, the calibrations that map the geometric location of the X-ray source with respect to a sensor or detector are performed with each 2D image.

In some known calibration processes, a fiducial marker containing one or more small balls or spheres is positioned somewhere in the field of view for each 2D X-ray projection image. This marker is placed so that each of the sphere(s) is part of a calibrated arrangement of spheres, where the distance from one sphere to the next is known to a high degree of precision relative to the pixel size of the 2D image. In the methods for accomplishing calibration through such arrays, the calibration device is assembled and then the device is calibrated (the distances from one sphere to the next are determined) based on an expensive and time-consuming CT imaging process using a high-resolution X-ray tomographic imaging system. This approach is required because it is not practical to assemble the spheres in their positions with a known accuracy on the level of microns (1 millionth of a meter), so the assembly must be calibrated after it has been assembled. A data file that notes the location of each object in the calibration array is then developed, linking that to the X-ray source and detector combination to describe the locations of the spheres in the array. As the treatment of dental patients often involves the use of several alignment aids to image the teeth, the requirement of a unique calibration array for each device presents possibilities for error when used, and this complicated method of calibration significantly raises the costs of the dental procedure.

### SUMMARY

This application relates generally to X-ray devices and systems. In particular, this application describes a calibration apparatus for an X-ray imaging system that comprises a housing containing an X-ray sensor and a calibration array having multiple calibration targets located within or on a substrate. Each calibration target can contain an element with an X-ray absorption different (either higher or lower) than that of the substrate. The multiple targets are in a fixed spatial relationship relative to each other. The calibration apparatus can be incorporated into an aligner and/or sensor-positioning device that is used with the X-ray imaging system. The calibration apparatus increases the accuracy of, and enables, the reconstruction of a three dimensional (3D) image from a series of two dimensional (2D) images taken by the X-ray imaging system.

### BRIEF DESCRIPTION OF THE DRAWINGS

The following description can be better understood in light of the Figures which show various embodiments and configurations of the imaging systems and methods. Together with the following description, the Figures demonstrate and explain the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

FIGS. 1A and 1B show a top schematic view of some embodiments of a 3D dental intra-oral imaging system for use with geometric calibration systems.

FIG. 2 shows a schematic view of some embodiments of a 3D dental intra-oral imaging system for use with geometric calibration systems.

FIG. 3 shows an operational schematic view of the imaging operation of a 3D dental intra-oral imaging system for use with geometric calibration systems.

FIGS. 4A through 4L show schematic views of calibration targets according to some embodiments.

FIG. 5 shows a side view of some embodiments of an aligner that aids in positioning the sensor, and a sensor housing that can be used with calibration apparatus that can be used in 3D dental intra-oral imaging systems.

FIG. 6 depicts a schematic view of some embodiments of an aligner and sensor housing that can be used with a calibration apparatus that can be used in 3D dental intra-oral imaging systems.

FIG. 7 shows a schematic view of other embodiments of an aligner and sensor housing that can be used with a calibration apparatus that can be used in 3D dental intra-oral imaging systems.

FIG. 8 shows a side view of other embodiments of an aligner that can be used with a calibration apparatus that can be used in 3D dental intra-oral imaging systems.

FIG. 9 illustrates some embodiments of a calibration apparatus containing a calibration object with an array of calibration targets.

FIG. 10 illustrates other embodiments of a calibration apparatus containing a calibration object with an array of calibration targets.

FIG. 11 shows a side view of some embodiments of an aligner that can be used with a calibration apparatus used in 3D dental intra-oral imaging systems.

FIG. 12 shows a view of some embodiments of a calibration pattern as an array of circles in an empty image field for clarity, and then with a second empty image showing how the calibration pattern is superimposed on the image and then subtracted out.

FIG. 13 shows a view of the calibration pattern of FIG. 12 as an array of circles with an actual image showing how the calibration pattern is superimposed on the image and then subtracted out.

### DETAILED DESCRIPTION

The following description supplies specific details in order to provide a thorough understanding. Nevertheless, the skilled artisan will understand that the described X-ray systems can be implemented and used without employing these specific details. Indeed, the described systems and methods can be placed into practice by modifying the described systems and methods and can be used in conjunction with any other apparatus and/or techniques conventionally used in the industry. For example, while the description below focuses on imaging systems using X-rays, other forms of electromagnetic or atomic radiation could be used, including gamma rays, neutron radiation and infra-red or visible light, depending on the absorptive characteristics of the objects to be imaged by the desired radiation. Ultrasonic energy could also be used with the appropriate sources and detectors to generate 3D images in a similar manner. In another example, while the description below focuses on dental X-ray equipment, other system configurations that hold the detector in a substantially fixed position while the X-ray source is moved around the object during an imaging scan, or conversely the X-ray source is held in a substantially fixed position while the detector is moved, could be used for dental 3D imaging or other 3D imaging applications.

In addition, as the terms on, disposed on, attached to, connected to, or coupled to, etc. are used herein, one object (e.g., a material, element, structure, member, etc.) can be on, disposed on, attached to, connected to, or coupled to another object—regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

Dental X-ray radiography can be performed by positioning an X-ray source on one side of an object (e.g., a tooth, a set of teeth, a portion or all of the jaw) and causing the X-ray source to emit X-rays through the teeth, soft tissue, and bone toward an X-ray detector (or sensor) located on the other side of the teeth, either inside or outside of the mouth. As the X-rays pass through the teeth, jaws, and other tissues, their energies are absorbed to varying degrees depending on the tissue composition and the total thickness of the tissues. The X-rays arriving at the X-ray detector form a 2D X-ray image based on the cumulative absorption through the teeth, bones and other mouth structures. These intraoral X-ray images provide a high level of detail of the tooth, bone, and supporting tissues. The images also allow users (i.e., dentists) to find cavities, examine tooth roots, evaluate the condition of the bony area around the tooth, determine if periodontal disease is present or a concern, and monitor the status of developing teeth, among other things.

Panoramic imaging, a popular form of extra-oral imaging, visualizes the entire maxilla, mandible, temporomandibular joints (TMJ) and associated structures in a single image, but it is subject to considerable geometric distortion and has relatively low spatial resolution compared with intraoral radiography. Again, this technique is limited because it provides a 2D representation of a 3D object. The 2D image results in superposition of overlying and underlying structures and the loss of all spatial information in the depth (or Z-axis) dimension. Consequently, important dimensional relationships are obscured, observed sharpness is reduced, objects of interest can be hidden, and pathology contrast is reduced. Accordingly, 3D imaging can be beneficially used in some dental procedures. Tomosynthesis is one type of 3D imaging that provides 3D information that is reconstructed from X-ray images of the patient taken from multiple perspectives within a scan angle that is much smaller than the 360° or at least 180° of computed tomography (CT) or cone-beam computed tomography (CBCT). Typical angular ranges for tomosynthesis cover a range from a minimum of perhaps 10° to perhaps an upper limit of 60° or 70°, depending on the application. As examples from 3D mammography, where the angular range sweeps over a portion of a circular arc, the Hologic Dimensions system has a 15° angular sweep, while the GE SenoClaire sweeps over 25° and the Siemens Inspiration sweeps through 50°. Digital tomosynthesis improves the visibility of anatomical structures by reducing visual clutter from underlying and overlying anatomy, typically portraying the 3D information in a series of image slices pulled from the 3D volume data.

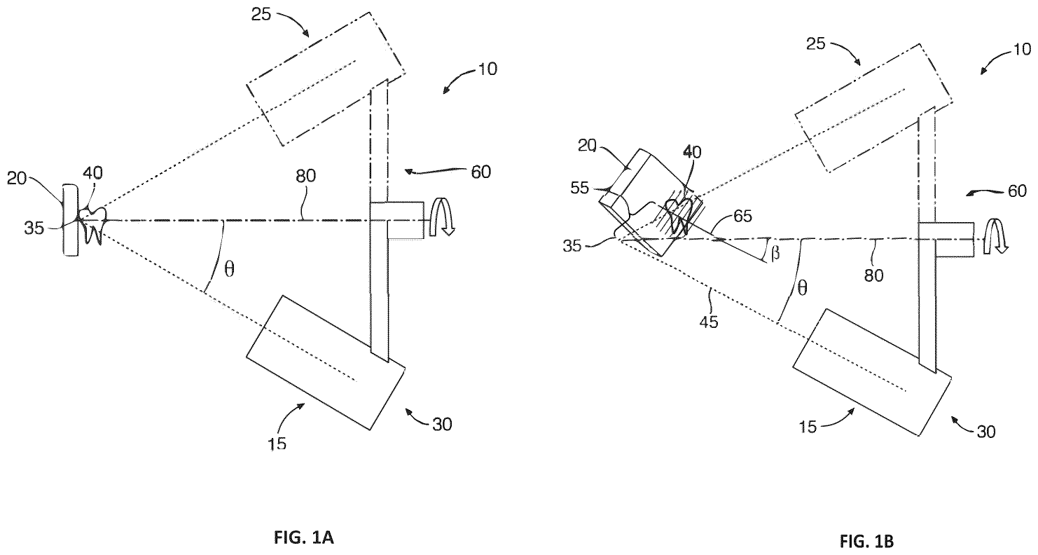

Some embodiments of a calibration apparatus used in a 3D X-ray imaging system are illustrated in the figures. FIG. 1A shows a 3D intra-oral X-ray image photography system 10 having X-ray source 30, and according to some embodiments an additional X-ray source 25. These X-ray source(s) are positioned around axis 80, which passes through imaging detector 20 and image target 40 to a center of rotation 35. In intra-oral embodiments, image target 40 is often a tooth or some other oral feature under examination. The X-ray source(s) include a housing affixed to a support arm 60. An angle θ between the axis 80 and X-ray source 30 defines both the X-ray source location and the X-ray source path according to embodiments having a fixed support arm 60.

FIG. 1B depicts a shot-to-shot variance that is compensated for by a geometric calibration mechanism according to at least some embodiments presented herein. As a result of such variance, the alignment of the X-ray source 30 is not perfect with respect to either the tooth 40 or the detector 20, such that the center of rotation 35 is not centered on the detector 20 but is offset by a distance 55. The axis 65 (that is normal to the detector 20) is aligned at an angle β from the axis of rotation 80. These variances are illustrative of the types of variances that are of concern, but are not a complete summary of all the possible types of variance that can occur. Another variance that could be determined or checked by calibration would be a variance in the length of the axis 80, for example. As a result of these, or other possible variances that can occur singly or in combination, the alignment of the rotation axis 80 and the location of the center of rotation 35 with respect to the tooth 40 and the detector 20 must be determined using calibration techniques. In other words, the purpose of the calibration technique is to accurately determine the spatial or geometric relationship or positioning between the X-ray source 15 and/or X-ray source 25 with the detector 20. This calibration technique must be applied to each image acquired since the X-ray source moves due to rotation around the axis 80 between the acquisition of each image.

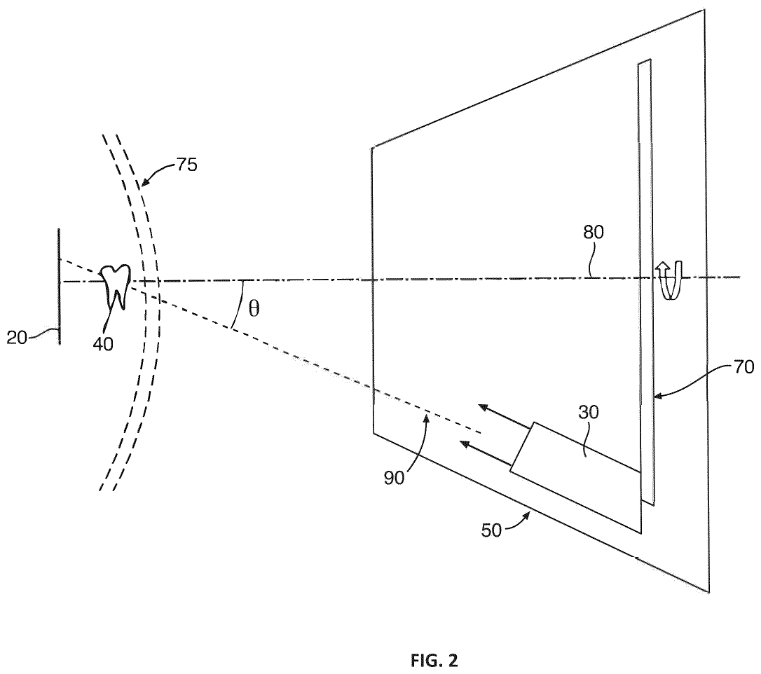

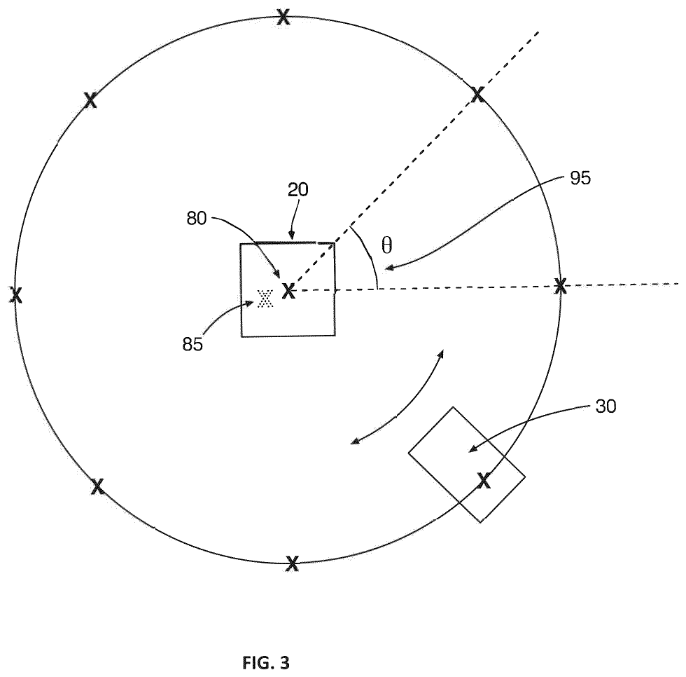

As depicted in FIGS. 2-3, an X-ray source 30 rotates in a circle in a plane that is roughly parallel to the X-ray detector and located on just one side of the patient's head. The detector 20 is stationary behind the tooth and in some configurations substantially parallel to the longitudinal axis of the tooth. So the X-ray source 30 can be rotated around an axis of rotation 80 that is approximately perpendicular to the plane of the detector so that the X-ray source itself rotates in a plane approximately parallel to the plane of the detector using mechanical gantry 70. As shown in FIG. 3, the X-ray source 30 is rotated around the axis of rotation 80 at a rotational velocity V that is not necessarily constant. The angle 95 shows the angular displacement between image acquisition points (“X”) that are arranged evenly around the circle. There may be any number of images taken. The position of each image acquisition point is determined by dividing the circle into a number of equal segments to provide the number of images desired. Alternatively, the image acquisition points may not all be distributed equally on the circle, but a substantial portion of the total number of images desired must be distributed around a substantial portion of the circle so that a number of images are obtained at angles 95 that are significantly different for each image obtained.

The X-ray source 30 does not necessarily physically stop for the image acquisition, but may be configured to emit a short pulse of X-rays at each image acquisition point. The X-ray source 30 may also be momentarily paused at each image acquisition point if X-ray source motion during the exposure is judged to be undesirable. The axis of rotation 80 can be centered on the detector 20 and may be substantially parallel to the detector normal. Since this will be difficult to fully achieve in practice due to challenges such as fitting the sensor into the patient's mouth given natural variations in anatomy and mouth size from patient to patient, a reconstruction algorithm can incorporate correction methods to enable a quality reconstruction even with some error in the orientation of the axis of rotation 80 or other errors from the ideal reconstruction geometry.

In some configurations, the axis of rotation 80 is not necessarily fixed on the center of the detector 20, but may be displaced from the center as depicted by the alternate location 85 of the axis of rotation 80. Indeed, the X-ray source 30 can rotate through any portion of the circular arc that ranges from 0 to about 360 degrees in that plane which is substantially vertical if the patient's tooth is oriented in a vertical direction and the detector is aligned with the tooth. Using the X-ray systems disclosed herein, the operator could conceivably take 2D images from any location in the parallel plane by changing the location in polar coordinates, theta and phi, rather than X and Y Cartesian coordinates. Alternatively, this same set of locations could be described using circular coordinates in the plane consisting of the radius R and the angle theta. Unlike some conventional tomosynthesis for mammography or chest X-rays where the source moves in a limited arc within a plane that is substantially perpendicular to the face of the detector, the present systems are not constrained in these ways.

The accuracy of these 3D X-ray imaging systems can be improved using a calibration apparatus. In order to reconstruct 3D images using tomosynthesis techniques or other mechanisms for assembling 3D images, it is important to know where an X-ray source is on a path in relation to the X-ray detector, or vice versa. While many of these paths are predetermined, small variances in patient/teeth location result in minor deviations from the desired path on a frame-to-frame basis. These errors compound during a tomosynthesis process, resulting in a compounded loss of quality in a final 3D image assembled from the 2D X-ray images, or may even result in a failure to generate a final 3D image if the overall errors are sufficiently large. In order to account for these small variances in the detector (or source) position, the geometric location of the X-ray source (or detector) can be independently calibrated with respect to the detector (or source) for each 2D X-ray image.

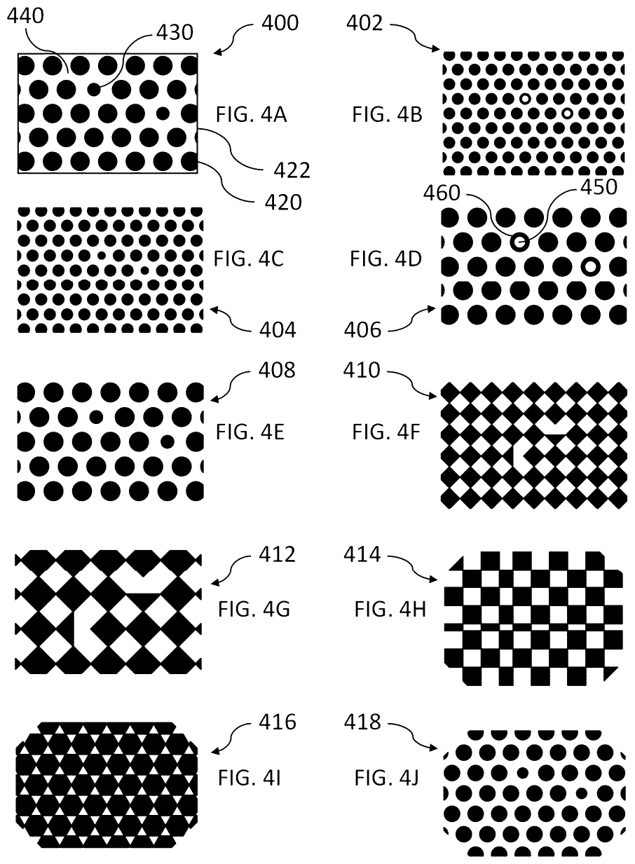

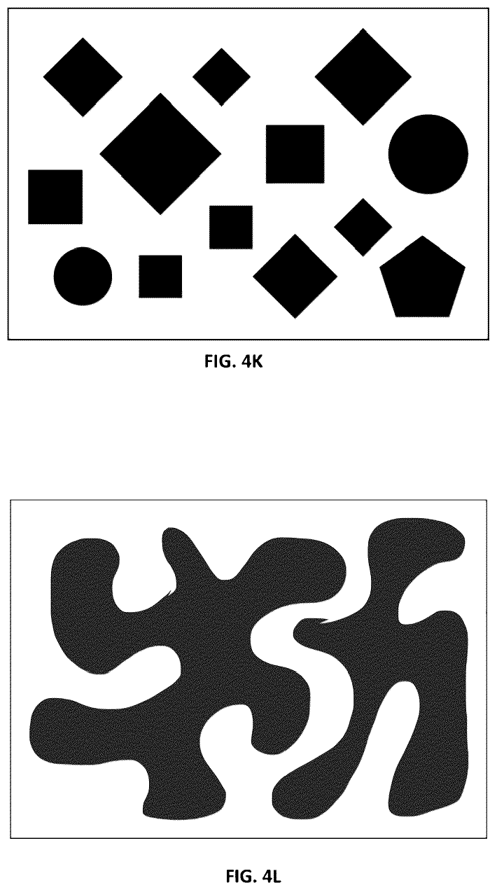

Some embodiments of the calibration apparatus that can be used in 3D imaging systems are depicted in FIGS. 4-13 and described herein. In these embodiments, the calibration apparatus and associated calibration algorithm can operate in tandem to minimize errors in the individual 2D images that will adversely impact the 3D image construction process.

In some embodiments, the calibration apparatus comprises an array of multiple calibration targets (or fiducial markers). In some embodiments, the calibration array can be made up of at least two calibration targets. In such embodiments, each target of the at least two targets can be formed in the shape of a circle, rectangle, rhombus, pentagon, hexagon, octagon, and/or polygon. The shape of the targets may be homogenous across or within the calibration array, or the shape of the targets may differ across or within the calibration object. In some configurations where a calibration array involves multiple tessellated targets, these targets can be separated by negatives of the targets themselves, or by other tessellated shapes. A variety of target shapes could be used at different points in the array.

In some embodiments, numbers and/or alphanumeric symbols could be incorporated into the targets of the array. As well, any kind of digital coding scheme could be applied in designing the targets of the array so that the digital code (such as a serial number or other useful information) could be provided along with the ability to provide the position data required for a geometric calibration. In some configurations, the digital coding scheme can comprise a barcode or a QR code. Encoding information, such as a serial number or some means of identifying either or both of the sensor and the sensor holder, could enable automatic pairing of the sensor with image correction information related to sensor characteristics or performance, or could be used to authenticate a sensor, or for other technical and/or business purposes.

FIGS. 4A-L depict several possible examples of calibration targets in an array 400. In the embodiments shown in FIG. 4A, the calibration array 400 has a plurality of targets 420 and 430, separated by blank space 440. In the embodiments of FIG. 4A, the targets 420 are larger than targets 430, even though both targets have similar circular cross-sectional shape. In the embodiments of FIG. 4B, the calibration array 402 has a plurality of targets of differing shapes and sizes. In the embodiments of FIG. 4C, the calibration array 404 is similar to calibration array 400, but with a tighter spacing between the targets. The embodiments of FIG. 4D show a calibration array 406 where the target 460 utilizes a second material or open space at its center, depending upon the desired configuration. The embodiments in FIG. 4D are similar to those depicted in array 402 of FIG. 4B, but with a wider grouping of targets and larger targets. FIG. 4E depicts some embodiments of a calibration array 408 that is similar to calibration array 400 of FIG. 4A, but lacking border 422. In the embodiments of FIG. 4F, the calibration array 410 utilizes multiple tessellated targets with slight variations near the center of the array. This configuration assists the operator in placing the array on an image or to assist in determining the orientation of the array since when the image is rotated, the orientation of the array is different from the un-rotated orientation. The embodiments of FIG. 4G show a calibration array 412 with larger targets than the array 410 of FIG. 4F. In the embodiments of FIG. 4H, a calibration array utilizing multiple tessellated rectangular targets of varying dimensions are present in an overall octagonal shape. A similar configuration is also used in the embodiments 416 of FIG. 4I and 418 of FIG. 4J.

Additional examples of the calibration targets are shown in FIGS. 4K and 4L. In these examples, the calibration targets can have configurations that are more irregular. The configurations in FIG. 4K have a mixture of shapes and sizes. The configurations in FIG. 4L have more random shapes that are highly contrasted and somewhat fill the entire area. Other configurations could have a design with dots (or diamonds in quinconce) that all have a different random size or that may systematically range in size. Other configurations that can be used include regular and irregular tiling design (such as Penrose tiling) and/or any pattern that will give a unique matching of rotation, scale, and/or translation. In the embodiments illustrated in FIGS. 4A-L, while only array 400 shows a border, borders of other shapes and sizes can be applied to any embodiments of the illustrated calibration arrays. In such embodiments having borders, the borders may utilize materials that have thicknesses that vary in relation to the targets of the calibration array. Additional arrays combining parts, combinations, or sub-combinations of the arrays and/or targets illustrated in FIGS. 4A-L can also be used.

It is helpful to realize that the embodiments illustrated in FIGS. 4A-L also depict different geometric arrangements of the targets. For example, FIGS. 4A through 4E all show a hexagonal arrangement of the targets, while FIGS. 4F through 4H show a rectangular arrangement of the targets. Other arrangements of the targets are also possible, such as a circular or ellipsoidal pattern, or the targets may be arranged in a random pattern. These geometric arrangements that can be used may have advantages. For example, a hexagonal arrangement provides three different symmetry axes whereas a rectangular pattern typically has four different axes of symmetry (the two diagonals and the two axes perpendicular to the “faces” of the rectangle), which may be useful in crafting the software algorithm that interprets the calibration target image during the calibration process. Other arrangements which could have similar benefits are also possible.

Another helpful characteristic of the target shapes is to consider how they can be analyzed by a software algorithm. In many embodiments, it is desirable to utilize target shapes that can be easily analyzed to determine their center point and then to use the set of all the center points of the targets in the array for all future calculations to determine the calibration for that image. For this reason, targets shaped like circles are often preferred because their center point is fairly easy to determine algorithmically. Other shapes that could also be easily analyzed to determine their geometric center point could include squares, hexagons, diamonds, etc.

The calibration targets can comprise a material defining the edges/exteriors and interiors (or outside and inside) of each target shape. According to some embodiments, the exteriors and interiors of the target shapes need not be crafted from the same materials. According to some embodiments, the array comprises higher-Z elements such as copper, tungsten, tantalum, molybdenum, gold, or other higher-Z elements that are suitable for processing in a semiconductor process, or can be etched easily, or which have other desirable characteristics or behaviors in a manufacturing process. An alloy or mixture of such elements can also be used in some configurations. Advantageous criteria for a material include susceptibility to patterning in high-accuracy lithography and pattern-transfer processes, including wet etching pattern transfer, reactive ion etch (RIE) pattern transfer, laser etching, lift-off patterning, and other manufacturing processes achieving similar results. Other helpful criteria would include the ability to be laid down on a substrate in thin uniform layers using processes such as vacuum evaporation, sputtering, chemical vapor deposition, or other techniques known in the art of semiconductor processing, electronic circuit board processing, etc.

The advantages of higher-Z elements include a higher absorption for X-rays compared to lower-Z elements, enabling a comparatively thinner layer of higher-Z elements or material to create a pattern in the 2D projection image. While lower-Z elements can be used in some embodiments, they need to be imaged under X-ray illumination with sufficient contrast to be detected in a captured image using machine vision, artificial intelligence techniques or other ways of analyzing the image data to identify and properly analyze each element in the array as captured in the X-ray image.

In some configurations the contrast is neither too low nor too high. For example, if the contrast between the array elements is low, it will complicate the design and implementation of the algorithm that will detect the presence of the individual elements in the image because they will be difficult to distinguish algorithmically from the images of the teeth, the other elements of the object being imaged, and/or the background levels in the image. On the other hand, if the array elements are too thick or too absorptive of X-rays compared to the background area, the result may be a loss of detail in the image. This situation would occur, for example, when the X-ray absorption of the array element combined with the object(s) being imaged is together so high that some or all portions of the combined element plus object(s) have essentially all of the X-ray energy and there is no detail left in the image. For these reasons, the desirable X-ray absorption of the array elements for the X-ray energies typically used in dentistry of between about 55 kV up to about 70 kV, lies above a minimum of about 10% absorption, above about 15% absorption, or even above about 20% or about 25% absorption. The maximum absorption in some embodiments should be no more than about 60% absorption, or less than about 50% absorption, or even less than about 40% absorption, or perhaps even about 35% or about 30% absorption. In other embodiments, the absorption can be any range or combination of these amounts. The optimum absorption for the calibration pattern can also depend on the performance characteristics of other elements of the imaging system such as the dynamic range of the X-ray sensor and the X-ray spectrum produced by the X-ray source, so what is optimum for some embodiments of a 3D dental X-ray imaging system may not be optimal for other embodiments and other x-ray systems.

In some embodiments, the calibration targets can be created by etching into a substrate to create the lower-absorbing or higher-absorbing regions or shapes by altering the thickness of the substrate material to create a different X-ray absorption. It is also possible to etch away some of the surrounding substrate material to form the calibration targets, leaving them thicker than the surrounding substrate. All of the other characteristics (e.g., size, shape, material) of these higher-absorbing, or lower-absorbing, calibration targets would be substantially the same as described herein for the calibration targets. So while the calibration arrays depicted in FIGS. 4A through 4J are described as containing a different material on a substrate, the calibration arrays could be configured as holes in the substrate rather than as additions of some material. These holes could be fabricated by an etching technique, such as those commonly used in the semiconductor industry. As a further variation, the calibration array of holes could be created by an etching process to leave the array elements as thinner portions of the same substrate material (or as areas with the substrate material completely removed), again creating an X-ray image due to the difference in X-ray absorption between the array elements and the substrate supporting or containing the array.

Calibration array targets configured in this manner would appear as areas of less X-ray absorption, rather than more X-ray absorption, in the resulting X-ray images. But they would still be visible and identifiable, and could be used in the same manner to calibrate the geometry of the X-ray image. One example of these embodiments is depicted in FIG. 10. In these embodiments, the calibration object 1000 contains a substrate 1010 with an array of holes 1020 in the substrate 1010. In some configurations, such as those shown in FIG. 10, the holes 1020 extend all the way through the substrate 1010. In other configurations, the holes 1020 only extend partially through the substrate 1010. The array can comprise one or more calibration targets with a first size and one or more calibration targets with a second size or any of the other configurations shown in FIGS. 4A-J.

In some configurations, the size of the array of calibration targets can depend on the size of the X-ray detector to be used with the array. In other configurations, the calibration apparatus comprises arrays of calibrations targets that are often on the order of the size of the detector (i.e., the detector active area). In yet other configurations, though, the size of the array of calibration targets can extend beyond the edges of the active detector active area. Depending on the algorithm used to analyze the calibration array images, though, it may be desirable to have the calibration array targets not extend beyond the edge of the detector active area as any array element that extends beyond the detector edge will be truncated or cut at the edge and will therefore appear as only a partial array element in the X-ray images. Partial array elements may complicate the analysis and calibration calculations, making a calibration algorithm less reliable or more complex.

The sizes of the individual calibration targets can vary widely. They need to be small enough to have a significant number of targets in the image, but large enough that they are easy to detect. The size of the individual calibration targets (or array elements or elements) of the array can be chosen to provide sufficient elements in the array for a reliable and statistically robust calibration process, while also making the elements large enough to cover a sufficient number of pixels in the X-ray detector. In some configurations, it is desirable that the array elements typically cover more than 20 pixels, or 50 pixels, or 100 pixels, or 200 pixels in each X-ray image.

In some configurations, the sizes of the calibration targets can be substantially similar across the array. In other configurations, the sizes of the calibration targets can vary across the array. These latter configurations are helpful to obtain orientation information out of the array, as well as a geometric calibration. For example, when the shapes of the calibration targets are not symmetrical, they can be used to determine the orientations of the objects being analyzed by the X-ray systems, or even the orientation of the detector when taking the image.

In some configurations, the array of calibration targets can be incorporated in the housing that contains the X-ray sensor/detector. This could be done using any number of methods, depending on the exact fabrication techniques used to make the sensor housing. The manufacturing approach used should be self-calibrating or otherwise known to have a high degree of repeatability and/or accuracy such that the resulting calibration array does not need to be checked, confirmed, or calibrated by an imaging technique such as a CT scan or other imaging process. Thus, the resulting calibration device contains a calibrated array that does not require an expensive verification process for each individual array produced. In one example, if the sensor housing is produced in an injection-molding process, the mold for the housing may be designed to hold the array elements individually in place with high accuracy while the housing material is injected into the mold and allowed to set, thus locking the array elements into an accurate, known position.

In other configurations, the array of calibration targets can be incorporated into an object that is combined with—or held by—the housing containing the X-ray sensor/detector. It could also be placed into a sensor holder that is designed to hold and position the sensor in a desired location. This object (or calibration object) can be associated with the calibration algorithms so that the calibration object meets at least three requirements. First, it can be made so that its geometric characteristics are inherently known to a high degree of accuracy. Second, it can be manufactured out of materials that are dimensionally stable over time. And, third, the calibration object can be manufactured using a reasonable-cost, high-volume process.

In some embodiments, the calibration object can comprise any substrate with a substantially flat, fairly rigid, stable material comprising lower-Z elements. The lower-Z elements may include aluminum, oxygen, nitrogen, hydrogen, lithium, carbon, silicon, other lower-Z elements, or combinations thereof. These elements could be combined to produce substrate materials such as glass, silicon, aluminum or aluminum alloys, plastics, magnesium fluoride, aluminum oxide, and similar compounds. Other suitable substrates could be made of materials similar to those used in making electronic circuit boards, including low density materials, flame retardant materials FR-1 through FR-6 materials, flexible substrates including Kapton, and composite epoxy materials CEM-1 through CEM-5. In some configurations, the calibration device can manufactured in a flat sheet or in a round wafer format. In these configurations, the wafers can be patterned using any of a wide variety of lithography tools and pattern transfer tools that are known in the semiconductor processing industry, the electronic circuit board industry, or other similar manufacturing techniques, and then diced or sectioned into discrete calibration arrays.

In the lower-Z element substrate embodiments, these elements absorb less X-rays than the higher-Z elements making up the array of calibration targets. As a result, there is a high contrast between the array of calibration targets and the substrate, rendering the substrate comparatively transparent. To aid this transparency, the substrate can be comparatively thin in relation to the target array, such that the substrate is even less likely to absorb X-rays than in embodiments where the substrate is thicker. The issue of transparency of the calibration target and substrate is important because the intent of the calibration array is to impose a detectable pattern on the X-ray image of the target object such as a tooth, but not to impose such a strong contrast calibration pattern on the X-ray image, or to absorb so much of the incident X-ray photons, that the X-ray image of the object is strongly obscured or too faint. Too much contrast in the calibration pattern, or too much absorption in the substrate and calibration pattern, will negatively impact the final 3D image of the target object.

The substrate of the calibration object can be manufactured using semiconductor processing techniques or other micro-machining techniques. In some embodiments, the calibration object can be manufactured using any lithography process. The substrate could have the array of calibration targets embedded therein or on a surface of the substrate. The calibration array could be configured in a tessellated pattern, for example, in a checkerboard arrangement of squares or rectangles laid out in a rectilinear array. Other arrangements could be a hexagonal pattern or other patterns that have a useful geometric relationship. Depending on the algorithm employed to interpret the image data, an irregular, random, or pseudo-random distribution of the array elements is also possible. In some configurations, the targets of the array are uniform and homogenous in size and shape. In other configurations, though, the targets of the array contain different sizes and shapes at different locations in the array.

The calibration device can be incorporated into the sensor housing or into an associated sensor holder in any manner that keeps the substrate of the calibration device substantially stable in position relative to the sensor image plane during the X-ray imaging process. When the sensor housing or the sensor holder is manufactured using injection molding, 3D printing, or other similar volume manufacturing processes, it can be manufactured with a recess configured to accept the substrate containing the array of calibration targets. The recess and the substrate can be covered by a first piece of the housing that is glued, ultrasonically welded, or otherwise attached to the second piece. Thus, the calibration object (the substrate with any array of calibration of targets) is securely held within the sensor holder and protected thereby, minimizing any variation between the calibration array and the detector during X-ray analysis, and accordingly minimizing any calibration inconsistencies between images in a 2D image series.

FIG. 9 illustrates some embodiments of a calibration object that contains a substrate with an array of calibration targets located on or in it. In these embodiments, the calibration object 900 contains a substrate 910 with an array of calibration targets on a surface. In these embodiments, the array can comprise one or more calibration targets 920 with a first size and one or more calibration targets with a second size 930.

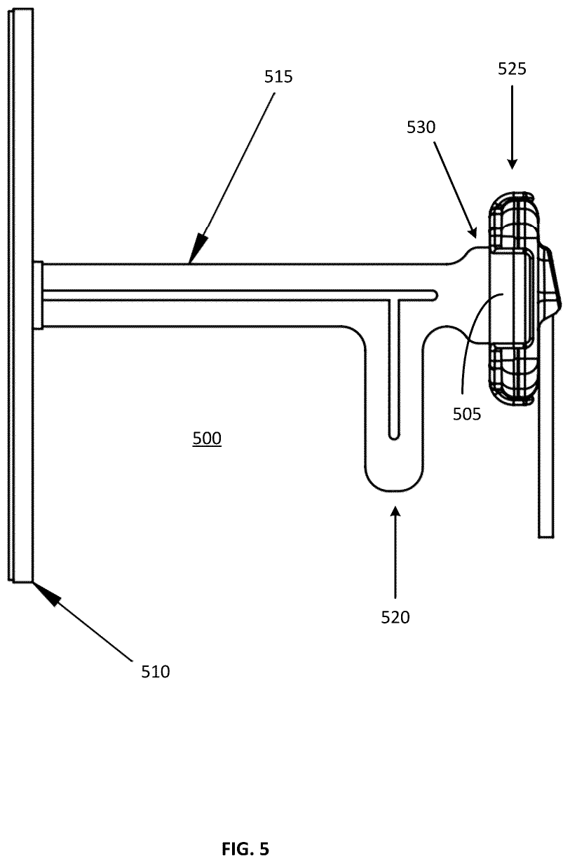

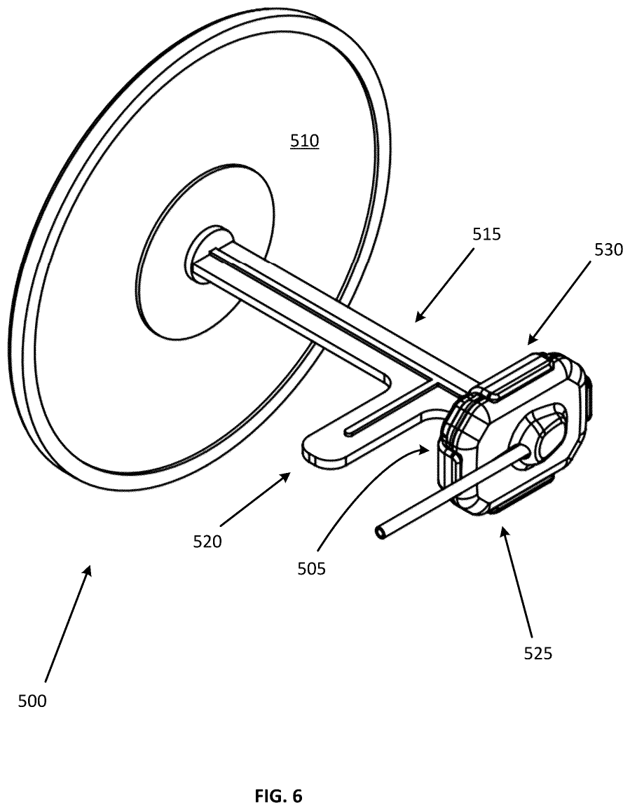

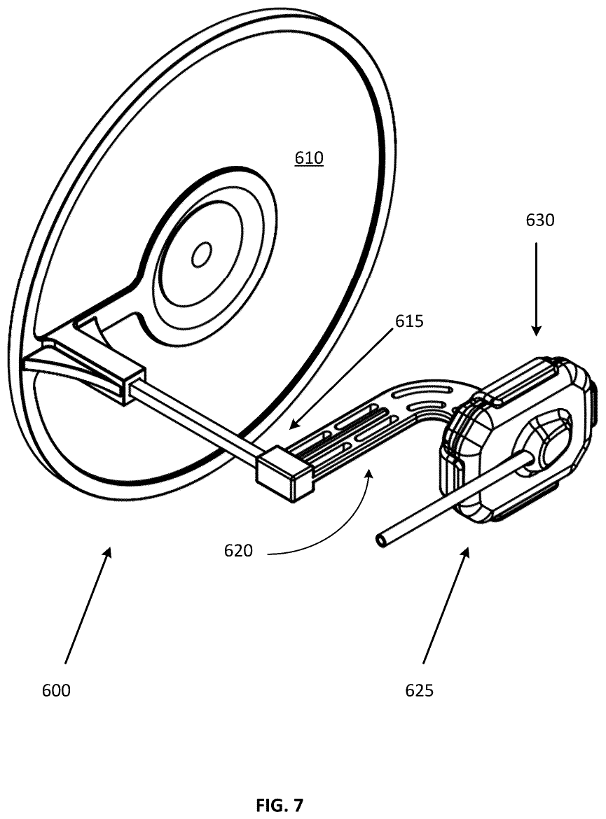

FIGS. 5 and 6 show some configurations of how the calibration object can be incorporated into the sensor housing. In these configurations, the sensor housing is part of aligner 500 that helps keep the X-ray sensor 505 substantially stationary and aligned behind the tooth of a patient while an X-ray image is taken. The aligner 500 contains a first portion 510 that can be attached to the X-ray head. It some embodiments, the first portion can contain a ferrous metal (or other magnetic material) that clips the aligner 500 into the X-ray head. The aligner 500 also contains a second portion 515 (or extender) that is orientated substantially perpendicular to the first portion and extends towards the patient's mouth. The aligner 500 also contains a third portion 520 that is connected to the second portion 515 at one end and at the other end is connected to the sensor holder 525. The sensor holder 525 contains a housing (or packaging) that is configured to hold the X-ray sensor 505, as well as the calibration object 530. In FIGS. 5 and 6, the extender 515 of the aligner 505 is configured to be connected to the middle of the first portion 510 of the aligner 500.

In other embodiments, as shown in FIG. 7, the extender 615 of the aligner 600 can be located near the edge of the first portion 610. In these embodiments, the third portion 620 has a curved configuration so that the sensor holder 625 (and the sensor 605) are aligned with the middle of the first portion 610. In some configurations, the first, second, and third portions of the aligners in FIGS. 5-7 can also be made separate and combined together to create the aligner. In other configurations, though, the first, second, and third portions can be manufactured as a single component to create the aligner.

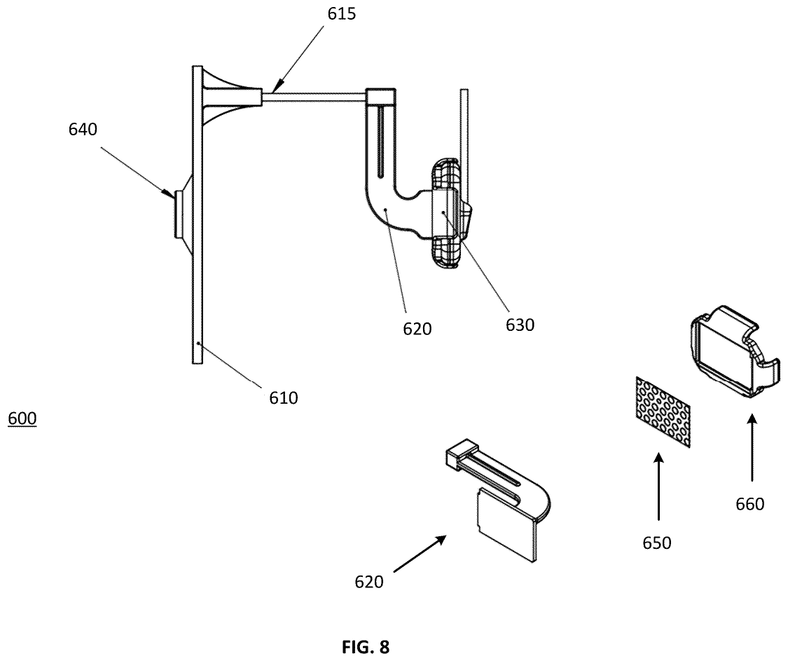

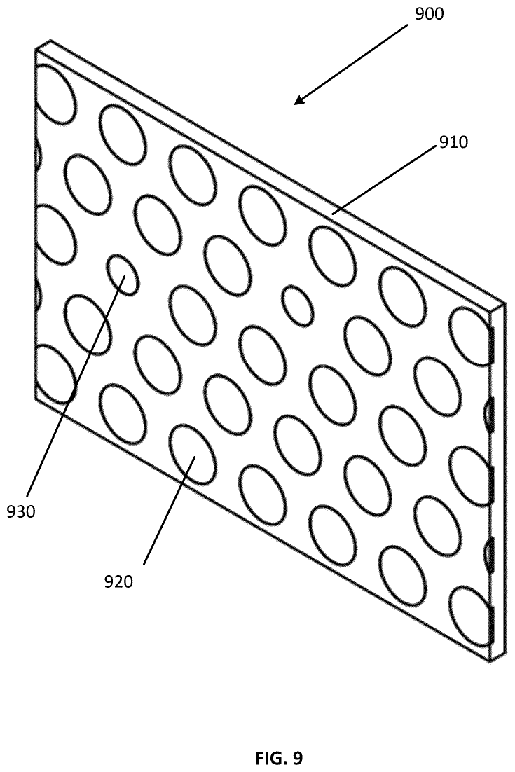

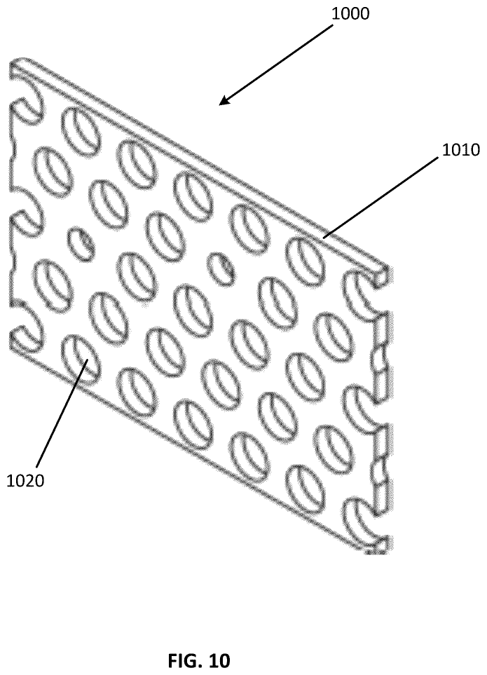

FIG. 8 illustrates some embodiments that show how the calibration object fits within the sensor housing. In these embodiments, the third portion 620 of the aligner 600 is attached to the sensor housing 660 so that the calibration object 650 is contained between them in a recess of the sensor housing. The calibration object 900 contains the substrate 910 with the array of calibration targets 920, 930, as described herein and illustrated in FIG. 9. The calibration object 1000 contains the substrate 1010 with the array of calibration targets 1020 as described herein and illustrated in FIG. 10.

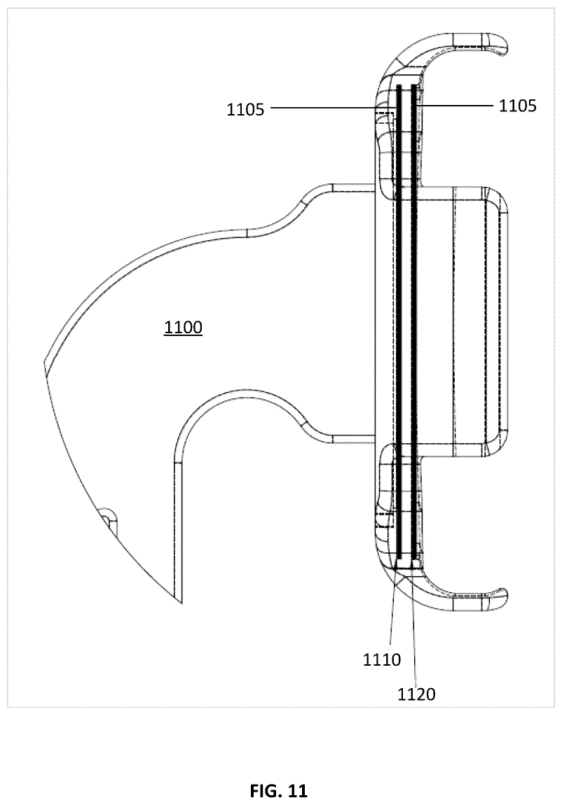

In other embodiments, the calibration object can be placed in other parts of the aligner instead of the sensor housing. For example, the calibration object could be placed in the second portion of the aligner since it is still oriented substantially parallel to the sensor. In another example, the calibration object could be placed in the first portion of the aligner since it is still oriented substantially parallel to the sensor. An example of these latter embodiments is depicted in FIG. 11. In FIG. 11, the calibration object 1105 could be placed in either the first position 1110 and/or the second position 1120 of the aligner 1100. In yet another example, the calibration object could be placed in any portion of the aligner that is oriented substantially parallel to the sensor.

The calibration object can be placed various distances away from the aligner and/or the sensor housing so long as it is located substantially parallel to the plane of the sensor. The calibration object should be placed close to the image plane of the sensor to keep the dimensions of the overall system as small as possible. But it can't be placed too close because a minimum distance is required between the pattern of the calibration object and the image plane of the detector to get the necessary parallax and geometry information. In some embodiments, the calibration object can be located less than about 1 cm away from the sensor. In other embodiments, the calibration object can be located about 1 mm to about 7 mm away from the sensor. In yet other embodiments, the calibration object can be located about 4 mm away from the sensor. In still other embodiments, this distance can be a range or combination of any of these amounts.

The calibration object is manufactured in such a way that its geometric characteristics (both of the substrate and the array of calibration targets) are known to a high-degree of precision. In some configurations, this known precision can be on the order of microns. In other configurations, the degree of precision for the array of calibration targets can be on the order of, or smaller than, about one fifth of the pixel size, about one quarter of the pixel size, about one third of the pixel size, or about one half of the pixel size, of a 2D image captured by the X-ray system. Thus, the relative positions of each target within the calibration array are known within a margin of error that is measured in fractions of the pixel size of the X-ray sensor. For example, where the sensor pixel size is approximately about 20 μm, the error in the accuracy of position of each target of the calibration array is about 4 μm or less, up to about 10 microns or less. In other embodiments this precision can be any combination, sub-combination, or ranges of these amounts.

In the embodiments shown in FIGS. 4-13, the distance between the image plane of the detector and the calibration array improves the system's ability to detect variation in the position of the image of the array on the sensor from one X-ray exposure to the other. So in these embodiments, the calibration array can be included within the detector package (or sensor holder) itself. In the configurations sized specifically for intra-oral applications, this separation could be as small as perhaps about 1 mm or could be as large as about 10 mm. The separation distance should be chosen to try to optimize the appearance of the calibration pattern on the X-ray sensor image. Obviously, the larger the separation, the more the calibration pattern will move across the image between one X-ray 2D exposure and another. While it is desirable in some configurations to have a significant amount of motion of the calibration pattern on the image, too much motion is undesirable because it will complicate the calibration algorithm if the calibration pattern moves so far that a portion of the calibration pattern is no longer on the sensor and is lost from the captured image. A larger amount could be used in some configurations since the limit on the upper size is just the ability for the sensor package plus holder to be placed in the patient's mouth, as long as the calibration algorithm can deal with the challenge of having a portion of the calibration pattern disappear from some images. The limits on the range is effectively determined by the geometry and the requirements for sufficient sensitivity to small errors in the position of the detector and X-ray source. In some embodiments, this separation can include a distance of about 0.3 mm to about 6 mm. In yet other embodiments, this separation can range from between 0.4 mm and about 4 mm. In other embodiments this separation can be any combination, sub-combination, or ranges of these amounts. In these configurations, this sizing optimizes a calibration array to fall within the pixel array of a 2D image containing multiple teeth.

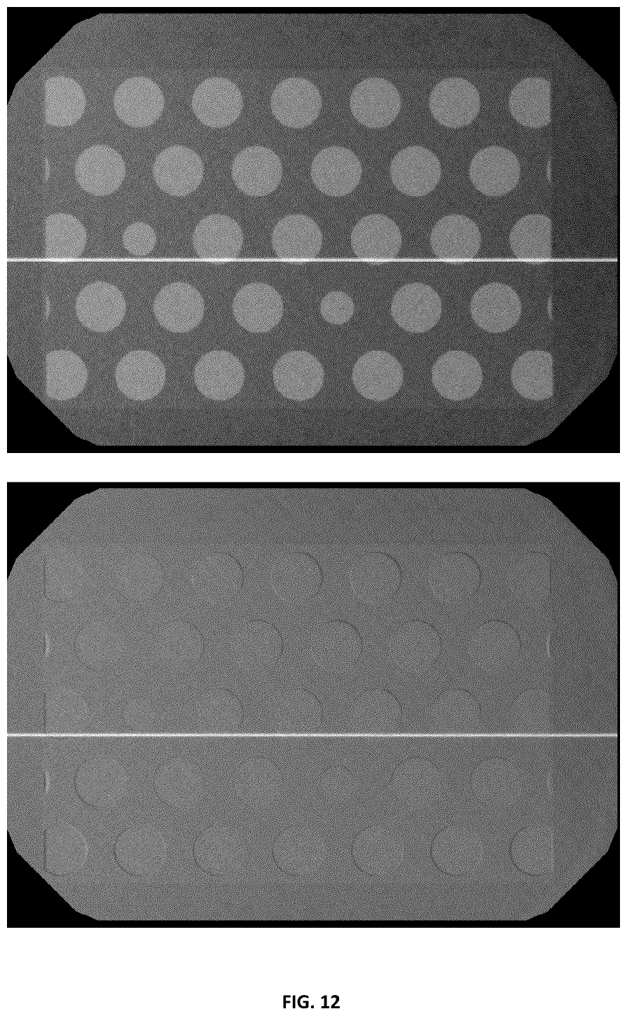

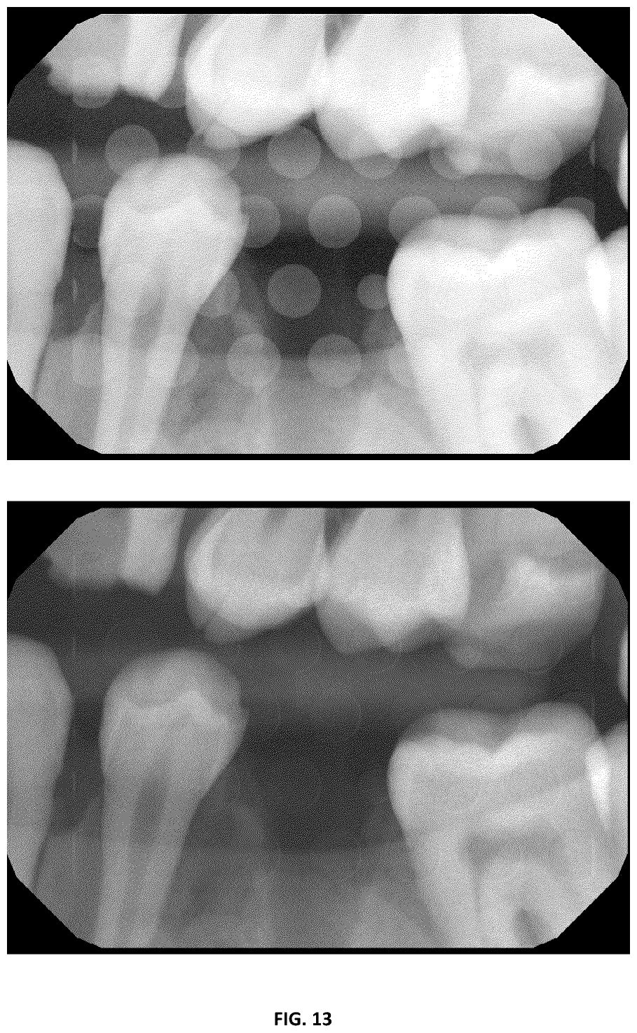

In the embodiments shown in FIGS. 4-13, the calibration apparatus is located in the aligner so that it will be located between the tooth of a patient and the detector. The calibration apparatus does not interfere with the imaging process for several reasons. First, since the reconstruction produces a 3D image, the region of interest (ROI) can be limited to the volume or 3D region that includes the teeth or other objects of interest, thus excluding the calibration array which will probably be at least a centimeter away from the object in most instances. Second, the imaging process and reconstruction algorithm can identify the array and each element then use that information to perform calculations to determine the geometry. So having a regular array can be helpful since the repetition of the elements' spacing and size can help identify the array of calibration targets in the image. After the geometry is determined, the reconstruction algorithm can remove or subtract the array of calibration targets from the 2D images before the 3D reconstruction is performed. An example of this removal can be seen in FIG. 12 between the upper and lower images. And third, as shown in the upper and lower images in FIG. 13, the material(s) in the calibration targets can be selected with a composition and thickness so that when the X-ray image of the array is combined with an X-ray image of the teeth, the array image is not too high of a contrast (i.e., too dark) but contains enough contrast so that the operator, or alternatively, the computer algorithm or reconstruction software, can see it, identify it, and successfully “remove” it without losing details on the teeth.

Using the calibration devices in the 3D X-ray systems as described herein provides several helpful features. One helpful feature is the improved image resolution and accuracy. Some conventional systems contain calibration mechanisms where the type of fiducial marker consists of a small ball or sphere that is positioned somewhere in the field of view for each 2D X-ray projection image so that each ball is part of a geometric arrangement of balls (i.e., vertices of a cube or rectangle, or other regular geometric shape such as a triangle or pyramid). The distance from one ball to the next and their relative locations to each other are typically known with an error that is measured in fractions of the pixel size of the X-ray sensor. In the case of calibrating an intra-oral X-ray image, the sensor pixel size is typically on the order of 20 μm, hence the error in the accuracy of position of the array of balls must be on the order of 5 μm or less. Producing such an accurately-positioned array in a volume-manufactured product is difficult and expensive because the best-known and most cost-effective manufacturing method would be to assemble the device using injection molding or other similar processes, and then to calibrate the array after manufacturing using a high-resolution 360 degree X-ray scan of the object. Such a scan would take several hundred separate images and then reconstruct the 3D image from this data. The X-ray scan of a single calibration array could take at least 15 minutes up to several hours to complete. This leads to high manufacturing costs and requires that each calibration object have a unique calibration data file that describes the location of the balls in the array. Maintaining the link between the data file and the specific calibration object significantly complicates the implementation of the system in actual use, incurring further costs and negatively impacts the reliability of the system given the realities of day-to-day usage in dental clinics. Since the treatment of any individual patient is expected to typically include the use of several alignment aids in order to properly image various teeth in particular positions in the mouth, this requirement that each alignment aid device be unique introduces unacceptable chances for error in use.

Another helpful feature is the improvement over some calibration systems that use a light diffraction grating. The calibration device described herein contains a pattern in both the X and Y directions, whereas a light diffraction grating only contains a pattern in the X direction. The use of a light diffraction grating requires that the grating be held external to the mouth so the diffraction pattern and/or grating can be seen by some type of sensor device, thereby complicating the use of the calibration technique and the use of the imaging system altogether. The calibration devices described herein, though, are not discernible to either the patient or the user and the process happens automatically and reliably without any user intervention or visual check to make sure that an element external to the mouth can be seen.

Indeed, the calibration apparatus are easier and simpler to use than some other calibration apparatus currently used for several reasons. First, the calibration systems described herein use easy algorithmic processing since they have a regular array containing the right target shapes with the array pattern having multiple axes of symmetry. Second, these calibration systems are inexpensive and mobile (from one X-ray system to another) since they have a pattern that is always the same in every unit produced so the system can assume the same pattern with the same dimensions and properties. This avoids the need to provide a unique calibration data file with each X-ray system unit. And third, these calibration systems keep the pattern contrast in a range where it is visible but does not have too much contrast. This makes the calibration algorithm simpler and more robust in obtaining the correct results every time.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. An X-ray imaging system, comprising:
an X-ray source;
an X-ray sensor oriented in a first plane; and
a calibration array oriented in a second plane substantially parallel to the first plane, the calibration array having multiple calibration targets located within or on a substrate, each calibration target comprising an element with an X-ray absorption different than that of the substrate, wherein the multiple calibration targets are in a fixed spatial relationship relative to each other.

2. The imaging system of claim 1, wherein the multiple calibration targets of the calibration array comprise a tessellated pattern.

3. The imaging system of claim 1, wherein the multiple calibration targets have a cross-section selected from circles, rectangles, rhombuses, pentagons, hexagons, octagons, polygons, or combinations thereof.

4. The imaging system of claim 1, wherein the calibration array is contained in an aligner for an X-ray source of the X-ray imaging system.

5. The imaging system of claim 1, wherein the calibration array is contained in a housing for the X-ray sensor.

6. The imaging system of claim 1, wherein the multiple calibration targets have a substantially similar shape.

7. The imaging system of claim 1, wherein the multiple calibration targets have cross-sections of a similar shape.

8. The imaging system of claim 1, wherein the multiple calibration targets are located on a surface of the substrate.

9. The imaging system of claim 1, wherein the multiple calibration targets are holes located within the substrate.

10. An imaging method, comprising:
using an imaging device to capture multiple 2D images, the imaging device comprising:
an X-ray source;
an X-ray sensor oriented in a first plane; and
a calibration array oriented in a second plane substantially parallel to the first plane, the calibration array having multiple calibration targets located within or on a substrate, each calibration target comprising an element with an X-ray absorption different than that of the substrate, wherein the multiple calibration targets are in a given spatial relationship relative to each other.
assembling the multiple 2D images into a three-dimensional (3D) image, wherein the location of each 2D image is calibrated using the calibration array.

11. The method of claim 10, wherein the multiple calibration targets of the calibration array comprise a tessellated pattern.

12. The method of claim 10, wherein the multiple calibration targets have a cross-section selected from circles, rectangles, rhombuses, pentagons, hexagons, octagons, polygons, or combinations thereof.

13. The method of claim 10, wherein the calibration array is contained in an aligner for an X-ray source of the X-ray imaging system.

14. The method of claim 10, wherein the calibration array is contained in a housing for the X-ray sensor.

15. The method of claim 10, wherein the multiple calibration targets have a substantially similar shape.

16. The method of claim 10, wherein the multiple calibration targets have cross-sections of a similar shape.

17. The method of claim 10, wherein the multiple calibration targets are located on a surface of the substrate.

18. The method of claim 10, wherein the multiple calibration targets are holes located within the substrate.

19. A calibration apparatus for an X-ray imaging system, comprising:
a substrate; and
a calibration array oriented in a plane substantially parallel to a plane of a sensor of the X-ray imaging system, the calibration array having multiple calibration targets located within or on the substrate, each calibration target comprising an element with an X-ray absorption different than that of the substrate, wherein the multiple calibration targets are in a known spatial relationship relative to each other.

20. The apparatus of claim 19, wherein the multiple calibration targets are located on a surface of the substrate or holes located within the substrate.
